(19) United States
(12) Patent Application Publication
(10) Pub. No.: US 2020/0283840 A1
(43) Pub. Date: Sep. 10, 2020

(54) EPIGENETIC DISCRIMINATION OF DNA

(71) Applicants: Ming-Che SHIH, (US); Academia Sinica

(72) Inventors: Ming-Che SHIH, Los Angeles, CA (US); Pao-Yang CHEN, Taipei (TW); Ming-Ren YEN, Taipei (TW); Fei-Man HSU, Taipei (TW); Yi-Jing LEE, Taipei (TW)

(21) Appl. No.: 16/325,097

(22) PCT Filed: Aug. 15, 2017
(86) PCT No.: PCT/US2017/046949

§ 371 Date: Feb. 12, 2019

Related U.S. Application Data
(60) Provisional application No. 62/375,358, filed on Aug. 15, 2016.

(51) Int. Cl. C12Q 1/6858; C12Q 1/6876; C12Q 1/686; C12Q 1/6869

(52) U.S. Cl. CPC C12Q 1/6858; C12Q 1/6876; C12Q 2600/154; C12Q 1/6869; C12Q 1/686

(57) ABSTRACT

The invention relates to methods of utilizing epigenetic information to separate one type of DNA from a mixture of multiple DNAs. The applications of the methods of the invention include, for example, the detection of chromosomal abnormality (e.g., aneuploidy, cancer cells), identification of genome abnormality, direct detection of DNA with abnormal copy number and development of indicators for the above-mentioned detection and identification.

### REFERENCE TO RELATED APPLICATION

This application claims priority to U.S. Provisional Application No.: 62/375358 filed Aug. 15, 2016, the disclosure of which is incorporated herein by reference in its entirety.

### FIELD OF THE INVENTION

The invention relates to a field of DNA discrimination. Particularly, the invention relates to methods of utilizing epigenetic information to separate one type of DNA from a mixture of multiple DNAs.

### BACKGROUND OF THE INVENTION

DNA methylation occurs after DNA synthesis by the enzymatic transfer of a methyl group from an S-adenosylmethionine donor to the carbon-5 position of cytosine. The enzymatic reaction is performed by a member of the family of enzymes known as DNA methyltransferases. The predominant sequence recognition motif for mammalian DNA methyltransferases is 5′-CpG-3′, although non-CpG methylation has also been reported. Approximately 50-60% of known genes contain clusters of CpG sites, known as CpG islands, in their promoter regions, and they are maintained in a largely unmethylated state, except in the cases of normal developmental gene expression control, gene imprinting, X chromosome silencing, ageing, or aberrant methylation in cancer and other pathological conditions. DNA methylation is tissue-specific and dynamic. The patterns of DNA methylation in the genome are a critical point of interest for genomic studies of cancer, epigenetic disease, early development, nutrition, and ageing. Methylation of DNA has been investigated in terms of cellular methylation patterns, global methylation patterns, and site-specific methylation patterns. The goal of methylation analysis includes the improvement of understanding cancer progression, and the development of diagnostic tools that allow the early detection, diagnosis, and treatment of cancers as well as other genomic diseases such as Down syndrome.

Epigenetics is the study of heritable changes in gene expression (active versus inactive) that do not involve changes to the underlying DNA sequence—a change in phenotype without a change in genotype. One major focus of epigenetic studies is the role of DNA methylation in silencing gene expression. Both increased methylation (hypermethylation) and loss of methylation (hypomethylation) have been implicated in the development and progression of cancer and other diseases. Hypermethylation of gene promoters and upstream coding regions results in decreased expression of the corresponding genes. It has been proposed that hypermethylation is used as a cellular mechanism to not only decrease expression of genes not being utilized by the cell, but also to silence transposons and other viral and bacterial genes that have been incorporated into the genome. Genomic regions that are actively expressed within cells are often found to be hypomethylated. Tumor suppresser genes are often found to be hypermethylated in cancer cells, compared to normal cells. Thus, there appears to be a cellular balance between silencing and expression of genes by hypermethylation and hypomethylation.

Bisulfite sequencing is one of the major experimental approaches to determine the methylation status of cytosines at a single nucleotide level. Briefly, single-stranded DNA is treated with sodium bisulfate, which sulfonates cytosine but leaves methylated cytosines unaffected. The cytosine is then deaminated and desulfonated to uracil [Frommer M, McDonald L E, Millar D S, Collis C M, Watt F, Grigg G W Molloy P L, Paul C L: A genomic sequencing protocol that yields a positive display of 5-methylcytosine residues in individual DNA strands. Proc. Natl. Acad. Sci. USA 1992, 89:1827-1831]. Bisulfite-converted DNA is amplified by PCR with appropriate primer pairs and PCR products are directly sequenced and aligned to unconverted DNA, thus revealing the methylation status of individual cytosines. To utilize the differential methylation pattern in DNA discrimination, US20050202490 uses sodium bisulfite to convert unmethylated cytosines to uracil followed by amplification with specific primers to determine DNA methylation status. The differential methylation of mixed DNAs could indicate that they are from different sources (e.g., parent and offspring, tumor and normal cells). To enrich specific DNA by the methylation pattern (hyper- or hypo-methylation), US20090203002 uses a methylation-sensitive restriction enzyme to digest sites with unmethylated CpGs followed by linker attachment, self-ligation, and circular amplification to amplify unmethylated DNA. WO2011082386 amplifies hypomethylated DNA by using a methylation-sensitive enzyme to digest sites with unmethylated CpGs followed by linker attachment, PCR amplification, linker removal, ligation of separate PCR to form a high molecular weight product, and amplification of this product by isothermal amplification. Together, these methods prove that differential patterns of DNA methylation may be used to discriminate specific DNAs in a mixture.

Several large genomic studies have indicated that the incidence of whole-chromosome aneuploidy in newborns is 1-2% [Hook E B, Rates of chromosomal abnormalities at different maternal ages, OBstet. Gynecol. 1981, 58:282-285; Wellesley D, et al., Rare chromosome abnormalities, prevalence and prenatal diagnosis rates from population-based congenital anomaly registers in Europe. European Journal of Human Genetics 2012, 20:521-526]. Such chromosome abnormalities represent a significant cause of prenatal morbidity and mortality as well as a major cause of severe developmental delay in long-term survivors. Given the maternal age dependence of common trisomies and the marked rise in average maternal age, it is clear that the importance of screening aneuploidy will continue to increase. Reliable, inexpensive, and non-invasive methods for the detection of aneuploidy during pregnancy are urgently needed. For example, Down syndrome is one of the most common chromosome abnormalities in humans, occurring in ˜1 per 800 newborns each year. The patients carry three copies of chromosome 21, rather than the normal two copies, and show severe intellectual disability. The need for long-term care causes a financial and emotional burden on the patients' families. Traditional invasive prenatal tests such as amniocentesis are highly accurate, but increase the risk of miscarriage. Amniocentesis is usually performed between 16-20 weeks of pregnancy. The demonstration that fetal cells of various lineages are present in maternal blood was an important milestone in prenatal testing. Approximately 10% of fetal DNA is found to exist in the maternal plasma between 10-21 weeks of pregnancy [Wang E. et al, Gestational age and maternal weight effects on fetal cell-free DNA in maternal plasma, Prenatal Diagnosis 2013, 33:662-666]; this low copy number of fetal DNA within the maternal plasma makes the identification of aneuploidy of individual chromosomes, specifically from fetal DNA, very challenging. Several techniques to purify such cells from maternal circulation were developed, and the feasibility of prenatal diagnosis of several conditions has been demonstrated. Nonetheless, such methods have not become practical, largely due to the paucity of fetal cells and the difficulty of their purification. Successful applications such as U.S. Pat. No. 8,563,242 provide methods to determine the aneuploidy based on calculating the ratio of the amount of a fetal methylated marker located on a target chromosome and the amount of a fetal genetic marker located on a reference chromosome. To further improve the signal to noise ratio (to enhance specific DNA that shows hyper/hypo methylation), US20120329667 uses a methylation-sensitive restriction enzyme (MSRE) to digest DNA from both test and control samples, followed by size selection to enrich the DNA with different DNA methylation regions. US20120315633 describes a method to enrich fetal nucleic acids from a cervical sample. These methods demonstrate the possibility of using DNA methylation patterns for DNA discrimination. However, as none of these methods can simultaneously detect methylated and unmethylated DNA, and none is designed to couple with next generation sequencing (NGS) technologies, they are still not applicable in genome-wide diagnosis, and are therefore very limited.

The purification of fetal cells from maternal circulation has been actively studied with very little success. The advancement in this respect can potentially greatly improve the detection of chromosomal abnormality for non-invasive prenatal testing and cancer diagnosis. Therefore, there is still a need to develop an improved detection of chromosomal abnormality.

### SUMMARY OF INVENTION

The invention provides a method to enrich one type of DNA from a mixture of two types of DNAs by their epigenetic signatures.

A significant application of the method is the detection of chromosomal abnormality (e.g., aneuploidy, cancer cells). In the follow-up application to identify genome abnormality, methods for directly detecting DNA with abnormal copy number and developing indicators are provided.

One aspect of the invention is to provide a method for detecting differentially methylated regions (DMR) comprising using one or more methylation-sensitive restriction endonucleases (MSREs) selected from the group consisting of Aor13HI, BspMII, AccIII, Aor51HI, Eco47III, BspT104104, AsuII, NspV, Eco52I, XmaIII, PluTI, PmaCI, PmlI and RsrII.

Another aspect of the invention is to provide a method for detecting polysomy in a test sample comprising fetal DNAs and maternal DNAs, comprising:
(a) isolating a DNA mixture from the test sample and a control sample;
(b) obtaining DNA fragments by digesting the DNA mixture with one or more methylation-sensitive restriction endonucleases (MSREs);
(c) amplifying specific differentially methylated regions (DMRs) by subjecting the DNA fragments to PCR amplification; and
(d) obtaining a ratio of the relative concentration of methylated fetal DNAs in the test sample to the relative concentration of methylated fetal DNAs in the control sample,
   wherein the relative concentration of methylated fetal DNAs in the test sample greater than that of the control sample indicates a likelihood of the presence of the polysomy in the test sample.

Another aspect of the invention is to provide a method for determining differentially methylated regions (DMRs) in genome-wide scale, comprising:
(a) isolating a DNA mixture from a test sample;
(b) generating an adapter-ligated DNA by ligating the DNA mixture with a sequencing adapter;
(c) obtaining a MSRE-digested DNA by digesting the adapter-ligated DNA with one or more methylation-sensitive restriction endonucleases (MSREs);
(d) obtaining PCR products by amplifying the MSRE-digested DNA with PCR;
(e) sequencing the PCR products by next generation sequencing (NGS); and
(f) determining DMRs in genome-wide scale.

In one embodiment of the invention, the method further comprises the step (g) of calculating the ratio of chromosome copy number of the test sample to the chromosome copy number of a control sample, wherein a ratio greater than 1.34 indicates a likelihood of the presence of polysomy in the test sample.

Another aspect of the invention is to provide a method for determining differentially methylated regions (DMRs) in genome-wide scale, comprising:
(a) isolating a DNA mixture from a test sample;
(b) obtaining DNA fragments by digesting the DNA mixture with one or more methylation-sensitive restriction endonucleases (MSREs);
(c) generating a biotin-ligated DNA by ligating the DNA fragments with a biotin-containing linker;
(d) enriching the biotin-ligated DNA with streptavidin beads;
(e) obtaining an adapter-ligated DNA by ligating the enriched biotin-ligated DNA with a sequence adapter; and
(f) sequencing the adapter-ligated DNA by next generation sequencing (NGS); and
(g) determining DMRs in genome-wide scale.

In one embodiment of the invention, the method further comprises the step (h) of calculating the ratio of chromosome copy number of the test sample to the chromosome copy number of a control sample, wherein a ratio greater than 1.34 indicates a likelihood of the presence of polysomy in the test sample.

Another aspect of the invention is to provide a method for determining differentially methylated regions (DMRs) in genome-wide scale, comprising:
(a) isolating a DNA mixture from a test sample;
(b) obtaining DNA fragments by digesting the DNA mixture with one or more methylation-sensitive restriction endonucleases (MSREs) wherein the unmethylated cytosines are present at the terminal nucleotides of the DNA fragments, and the methylated cytosines are present at the middle nucleotides of the DNA fragments;
(c) generating a sequencing adapter-ligated DNA by ligating the DNA fragments with a sequencing adapter;
(d) obtaining PCR products by amplifying the sequencing adapter-ligated DNA with PCR;
(e) sequencing the PCR products by next generation sequencing (NGS); and
(f) determining DMRs in genome-wide scale.

In one embodiment of the invention, the method further comprises the step (g) of calculating the ratio of chromosome copy number of the test sample to the chromosome copy number of a control sample, wherein a ratio greater than 1.34 indicates a likelihood of the presence of polysomy in the test sample.

Another aspect of the invention is to provide a method for determining differentially methylated regions (DMRs) in genome-wide scale:
(a) isolating a DNA mixture from a test sample;
(b) generating an adapter-ligated DNA by ligating the DNA mixture with a sequencing adapter;
(c) obtaining a sodium bisulfite-treated DNA by treating the adapter-ligated DNA with sodium bisulfite;
(d) obtaining PCR products by amplifying the sodium bisulfite-treated DNA with PCR; and
(e) sequencing the PCR products by next generation sequencing (NGS); and
(f) determining DMRs in genome-wide scale.

In one embodiment of the invention, the method further comprises the step (f) of calculating the ratio of chromosome copy number of the test sample to the chromosome copy number of a control sample, wherein a ratio greater than 1.34 indicates a likelihood of the presence of polysomy in the test sample.

In one embodiment of the invention, the polysomy is trisomy.

In one embodiment of the invention, the ratio is greater than 1.36, 1.38, 1.40, 1.42, 1.44, 1.46, 1.48, 1.19, 1.498, 1.50, 1.52, 1.54, 1.56, 1.58, 1.60, 1.65, 1.70, 1.80, 2.00, 2.2, 2.4, 2.6, 2.8, or 3.0.

In one embodiment of the invention, the ratio is greater than 1.46, 1.48, 1.498 or 1.50.

In one embodiment of the invention, when the ratio of the concentration of ratio of copy number of fetal DNA to total copy number of the DNA mixture is less than 10%, the method shows at least 13.5% improvement as compared to a method without the step of digestion.

In one embodiment of the invention, when the ratio of the concentration of ratio of copy number of fetal DNA to total copy number of the DNA mixture is less than 15%, the method shows at least 40% improvement as compared to a method without the step of digestion.

In one embodiment of the invention, the MSRE is selected from the group consisting of AatlI, AccII, FnuDII, AciI, AclI, AfeI, AgeI, Aor13HI, BspMII, AccIII, Aor51HI, Eco47III, AscI, AsiSI, AvaI, BceAI, BmgBI, BsaAI, BsaHI, BsiEI, BsiWI, BsmBI, BspDI, BspT104104, AsuII, NspV, BsrFI, BssHII, BstBI, BstUI, Cfr10I, ClaI, EagI, Eco52I, XmaIII, FauI, FseI, FspI, HaeII, HgaI, HhaI, HinP1I, HpaII, Hpy99I, HpyCH4IV, KasI, MluI, NaeI, NarI, NgoMIV, NotI, NruI, PaeR7I, PluTI, PmaCI, PmlI, PvuI, RsrII, SacII, SalI, SfoI, SgrAI, SmaI, SnaBI, TspMI and ZraI.

In one embodiment of the invention, wherein the MSRE is selected from the group consisting of Aor13HI, BspMII, AccIII, Aor51HI, Eco47III, BspT104104, AsuII, NspV, Eco52I, XmaIII, PluTI, PmaCI, PmlI and RsrII.

### BRIEF DESCRIPTION OF THE DRAWINGS

FIG. 1 refers to a schematic plot of the methods (Methods 1 to 5) of the invention.

FIG. 2 refers to the concept of Method 1 of the invention. Methylated DNAs are enriched and then quantified by quantitative PCR (qPCR). Each dot represents one nucleotide. The eight nucleotides composed of dark blue dots represent a restriction endonuclease recognition site and red dots represent methylated cytosines.

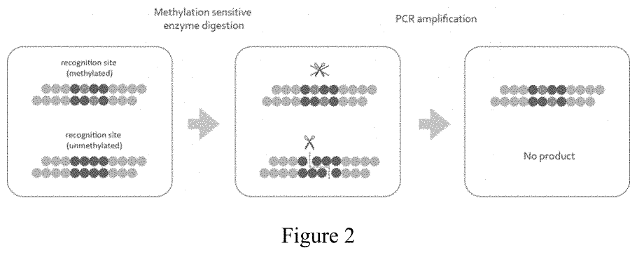

FIG. 3 refers to the electrophoresis result of PCR products undigested or digested by novel MSRE PmlI. The abbreviation “M” denotes “methylated test DNA.” The abbreviation “UM” denotes “unmethylated test DNA.”

FIGS. 4A (with MSRE digestion) and 4B (without MSRE digestion) refer to qPCR result of Method 1 validation.

FIG. 5 refers to the concept of Method 2 of the invention. Methylated DNAs are enriched and then quantified by quantitative PCR (qPCR). The eight nucleotides composed of dark blue dots represent a restriction endonuclease recognition site and red dots represent methylated cytosines. Yellow dots represent the Y-shaped sequencing adapter.

FIG. 6 refers to the electrophoresis result of PCR products undigested or digested by the MSRE PvuI. The abbreviation “M” denotes “methylated test DNA.” The abbreviation “UM” denotes “unmethylated test DNA.”

FIG. 7 refers to an image of the genome browser showing the abundance of methylated and unmethylated DNA reads from Method 2 of the invention.

FIG. 8 refers to the concept of Method 3 of the invention. Unmethylated DNAs are enriched and then sequenced by NGS. Each dot represents one nucleotide. The eight nucleotides composed of dark blue dots represent a restriction endonuclease recognition site and red dots represent methylated cytosines. Pink dots represent biotin-labeled linker. Yellow dots represent the Y-shaped sequencing adaptor.

FIG. 9 refers to the concept of Method 4 of the invention for post-sequencing identification of methylated and unmethylated DNA. Each dot represents one nucleotide. The eight nucleotides composed of dark blue dots represent a restriction endonuclease recognition site and red dots represent methylated cytosines. Yellow dots represent the Y-shaped sequencing adapter.

FIG. 10 refers to an image of the genome browser showing methylated and unmethylated DNA from Method 4 of the invention.

FIG. 11 shows the mechanism of bisulfite conversion. After bisulfite conversion and PCR, unmethylated-cytosines will be converted to thymines while methylcytosines remain unchanged.

FIG. 12 refers to an image of the genome browser showing the methylated and unmethylated DNA from Method 5 of the invention. The arrows indicate the sites showing differential methylation.

### DETAILED DESCRIPTION OF THE INVENTION

This invention aims to discriminate DNA from a mixture of multiple DNAs. Accordingly, the invention includes more than one method that utilizes epigenetic information to discriminate one type of DNA from a mixture. These methods are implemented significantly differently for various applications.

### Definitions

The following definitions are provided to facilitate understanding of the claimed subject matter. Terms that are not expressly defined herein are used in accordance with their plain and ordinary meanings.

Unless otherwise specified, “a” or “an” means “one or more.”

As used herein, the terms “individual,” “subject,” “host,” and “patient” are used interchangeably and refer to any mammalian subject for whom diagnosis or treatment is desired, particularly humans.

Often, ranges are expressed herein as from “about” one particular value and/or to “about” another particular value. When such a range is expressed, an embodiment includes the range from the one particular value and/or to the other particular value. Similarly, when values are expressed as approximations, by use of the word “about,” it will be understood that the particular value forms another embodiment. It will be further understood that the endpoints of each of the ranges are significant both in relation to and independently of the other endpoint. As used herein the term “about” refers to ±30%, preferably±20%, more preferably±10%, and even more preferable±5%.

As used herein, the term “polysomy” refers to a condition of presence of three or more copies of the chromosome rather than the expected two copies. Examples of polysomy include trisomy, tetrasomy, pentasomy, hexasomy, heptasomy, octosomy, nanosomy, decasomy and so forth.

As used herein, the term “trisomy” refers to a type of polysomy in which there are three copies of a particular chromosome, instead of the normal two. The most common types of trisomy in humans are trisomy 21 (Down syndrome), trisomy 18 (Edwards syndrome), trisomy 13 (Patau syndrome), trisomy 9, trisomy 8 (Warkany syndrome 2) and trisomy 22.

As used herein, the term “gene” indicates any gene of the family to which the named “gene” belongs, and includes not only the gene sequences found in publicly available databases, but also encompasses all transcript and nucleotide variants of these sequences.

As used herein, the term “genome-wide” refers to the entire genome of a cell or population of cells, or most or nearly all of the genome.

As used herein, the term “enrich” refers to the process of amplifying polymorphic target nucleic acids contained in a portion of a biological sample.

As used herein, the term “methylation state” or “methylation status” refers to the presence or absence of a methylated cytosine residue in one or more CpG dinucleotides within a nucleic acid.

As used herein, the term “differentially methylated regions (DMRs)” refers to genomic regions with different DNA methylation status across different biological samples.

As used herein, a biological sample refers to a sample, typically derived from a biological fluid, cell, tissue, organ, or organism, comprising a nucleic acid or a mixture of DNAs with different methylation patterns. The biological sample includes but is not limited to tissues, feces, hair, serum, plasma, skin, urine and whole blood.

As used herein, the term “biological fluid” refers to a liquid taken from a biological source and includes, for example, blood, serum, plasma, sputum, lavage fluid, cerebrospinal fluid, urine, semen, sweat, tears, and saliva. As used herein, the terms “blood,” “plasma,” and “serum” expressly encompass fractions or processed portions thereof. Similarly, where a sample is taken from a biopsy, swab, or smear, the “sample” expressly encompasses a processed fraction or portion derived from the biopsy, swab, or smear.

As used herein, the term “maternal sample” refers to a biological sample obtained from a pregnant female subject.

As used herein, the terms “maternal nucleic acids” and “fetal nucleic acids” refer to the nucleic acids of a pregnant female subject and the nucleic acids of the fetus being carried by the pregnant female, respectively.

As used herein, the term “fetal fraction” refers to the fraction of fetal nucleic acids present in a sample comprising fetal and maternal nucleic acid. Fetal fraction is often used to characterize the cell free DNA (cfDNA) in a mother's blood.

As used herein the term “chromosome” refers to the heredity-bearing gene carrier of a living cell that is derived from chromatin and comprises DNA and protein components (especially histones).

As used herein, the term “sequence of interest” refers to a nucleic acid sequence that is associated with a difference in sequence representation. A sequence of interest can be a sequence on a chromosome that is misrepresented, i.e. over- or under-represented, in a genetic condition. A sequence of interest may be a portion of a chromosome or an entire chromosome. A “test sequence of interest” is a sequence of interest in a biological sample.

As used herein, the term “adapter” is a short, chemically synthesized, single-stranded or double-stranded oligonucleotide that can be ligated to the ends of other DNA or RNA molecules. The term “adapter” may be a “sequencing adapter” used for sequencing the sequence of interest. A non-limiting example of the sequencing adapter is “Illumina Adapter Sequences,” which is available on the website https://support.illumina.com/downloads/illumina-customer-sequence-letter.html.

As used herein, the term “Next Generation Sequencing (NGS)” refers to sequencing methods that allow for high throughput parallel sequencing of clonally amplified molecules and single nucleic acid molecules. Non-limiting examples of NGS include sequencing-by-synthesis using reversible dye terminators, and sequencing-by-ligation.

As used herein, the term “altered amount” of a marker or “altered level” of a marker refers to increased or decreased copy number of the marker and/or increased or decreased expression level of a particular marker gene or genes in a biological sample, as compared to the expression level or copy number of the marker in a control sample. The term “altered amount” of a marker also includes an increased or decreased protein level of a marker in a sample, e.g., a cancer sample, as compared to the protein level of the marker in a normal, control sample.

Methods for Discriminating Specific DNA from a Mixture of Multiple DNAs

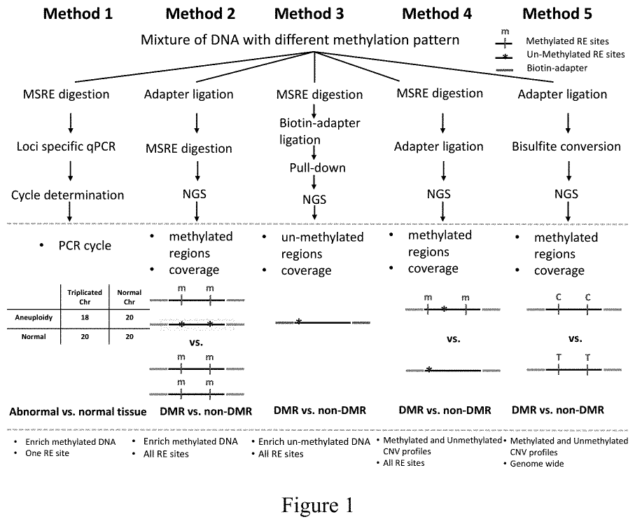

These methods use one or more novel MSREs to amplify methylated DNA(s) or use methylation differences in combination with NGS to analyze methylated and/or unmethylated sites in the whole genome. A schematic plot of these methods is shown in FIG. 1. Method 1 enriches methylated DNA of specific target sites, Method 2 enriches methylated DNA and performs genome-wide screening, Method 3 differentiates DNAs by their methylation patterns based on the location of the MSRE cutting site relative to the 5′ end of reads, and Method 4 segregates genome-wide methylation profiles to infer genomic copy number variation of DNA of different types.

In one aspect, the invention provides a method (Method 1) for enriching and detecting methylated DNA in a biological sample, comprising (a) isolating DNA from the sample, (b) obtaining DNA fragments by digesting the DNA mixture with one or more methylation-sensitive restriction endonucleases (MSREs), (c) amplifying specific differentially methylated regions (DMRs) by subjecting the DNA fragments to PCR amplification, and (d) comparing the relative concentration of methylated fetal DNAs in the test sample to the relative concentration of methylated fetal DNAs in the control sample, wherein the relative concentration of methylated fetal DNAs in the test sample greater than that of the control sample indicates a likelihood of the presence of the polysomy in the test sample. In one embodiment, the method further comprises obtaining a ratio of the relative concentration of methylated fetal DNAs in the test sample to the relative concentration of methylated fetal DNAs in the control sample, wherein the ratio greater than 1.34 indicates a likelihood of the presence of the polysomy in the test sample. In some embodiments, the ratio is greater than 1.36, 1.38, 1.40, 1.42, 1.44, 1.46, 1.48, 1.19, 1.498, 1.50, 1.52, 1.54, 1.56, 1.58, 1.60, 1.65, 1.70, 1.80, 2.00, 2.2, 2.4, 2.6, 2.8, or 3.0. In a further embodiment, the ratio is greater than 1.46, 1.48, 1.498 or 1.50.

In one embodiment, Method 1 is to enrich and detect methylated DNA in a biological sample, and comprises (a) isolating DNA from the sample, (b) digesting the DNA with one or more MSRE, or a combination thereof, (c) performing loci specific PCR amplification (such as qPCR) using primer pairs designed to amplify specific differentially methylated regions (DMRs), and (d) detecting the copy number of methylated DNA.

Any suitable methods known in the art can be used to isolate circulating cell-free fetal (CCF) DNA in the method. For example, a commercially available DNA extraction kit can be used in the isolation of DNA.

The isolated DNA can be digested to obtain DNA fragments with one or more methylation-sensitive restriction endonucleases (MSREs). According to one embodiment of the invention, the MSREs arelisted in Table 1.

TABLE 1

| Enzyme | Cutting site |
|---|---|
| AatlI | GACGT/C |
| AccII/FnuDII | CG/CG |
| AciI | CCGC(−3/−1) |
| AclI | AA/CGTT |
| AfeI | AGC/GCT |
| AgeI | A/CCGGT |
| *Aor13HI/BspMII/AccIII | T/CCGGA |
| *Aor51HI/Eco47III | AGC/CGT |
| AscI | GG/CGCGCC |
| AsiSI | GCGAT/CGC |
| AvaI | C/YCGRG |
| BceAI | ACGGC(12/14) |
| BmgBI | CACGTC(−3/−3) |
| BsaAI | YAC/GTR |
| BsaHI | GR/CGYC |
| BsiEI | CGRY/CG |
| BsiWI | C/GTACG |
| BsmBI | CGTCTC(1/5) |
| BspDI | AT/CGAT |
| *BspT104104/AsuII/NspV | TT/CGAA |
| BsrFI | R/CCGGY |
| BssHII | G/CGCGC |
| BstBI | TT/CGAA |
| BstUI | CG/CG |
| Cfr10I | R/CCGGY |
| ClaI | AT/CGAT |
| EagI | C/GGCCG |
| *Eco52I/XmaIII | C/GGCCG |
| FauI | CCCGC(4/6) |
| FseI | GGCCGG/CC |
| FspI | TGC/GCA |
| HaeII | RGCGC/Y |
| HgaI | GACGC(5/10) |
| HhaI | GCG/C |
| HinP1I | G/CGC |
| HpaII | G/CGC |
| Hpy99I | CGWCG/ |
| HpyCH4IV | A/CGT |
| KasI | G/GCGCC |
| MluI | A/CGCGT |
| NaeI | GCC/GGC |
| NarI | GGC/GCC |
| NgoMIV | G/CCGGC |
| NotI | GC/GGCCGC |
| NruI | TCG/CGA |
| PaeR7I | C/TCGAG |
| *PluTI | GGCGC/C |
| *PmaCI | CAC/GTG |
| *PmlI | CAC/GTG |
| PvuI | CGAT/CG |
| *RsrII | CG/GWCCG |
| SacII | CCGC/GG |
| SalI | G/TCGAC |
| SfoI | GGC/GCC |
| SgrAI | CR/CCGGYG |
| SmaI | CCC/GGG |
| SnaBI | TAC/GTA |
| TspMI | C/CCGGG |
| ZraI | GAC/GTC |

*indicates MSREs which were not reported in prior art references.

In Table 1, Aor13HI/BspMII/AccIII, Aor51HI/Eco47III, BspT104104/AsuII/NspV Eco52I/XmaIII, PluTI, PmaCI, PmlI, and RsrII are novel MSREs. Accordingly, the invention provides a method for detecting differentially methylated regions (DMR) comprising using one or more methylation-sensitive restriction endonucleases (MSREs) selected from the group consisting of Aor13HI, BspMII, AccIII, Aor51HI, Eco47III, BspT104104, AsuII, NspV, Eco52I, XmaIII, PluTI, PmaCI, PmlI and RsrII.

In one embodiment, the MSRE used in the method is AciI, BstUI, HhaI, HinPlI, HpaII or PvuI, or a combination thereof. In a further embodiment, the MSRE is a combination of AciI, BstUI, HhaI, HinPlI, HpaII and PvuI. In another embodiment, the MSRE is Aor13HI, BspMII, AccIII, Aor51HI, Eco47III, BspT104104, AsuII, NspV, Eco52I, XmaIII, PluTI, PmaCI, PmlI or RsrII, or a combination thereof.

In one embodiment of Method 1 of the invention, the circulating cell-free fetal (CCF) DNA is isolated and digested with MSREs (Table 1). PCR is performed using primer pairs designed to amplify fetal methylated regions (maternal unmethylated regions). The indicator of genome abnormality is shown in Table 2.

In one example of the method, with DNA enrichment as used in Method 1, the ratio of chromosome copy number between test chromosome and control chromosome in the normal sample is 2/22=0.091 (maternal DNA is digested) whereas the ratio of chromosome copy number is 3/22=0.136 in the trisomy sample (see Table 2 below). The ratio to discriminate between trisomy and normal sample is 0.136/0.0911.50. Comparing to the methods without enrichment, Method 1 provides improvement of (1.500-1.045)/1.045≅43.5% over previous approaches. Method 1 therefore significantly improves the resolution. Method 1 enhances signals of low levels of fetal DNA in plasma, therefore providing a possibility of early diagnosis.

In another aspect, the invention provides a method (Method 2) for selective amplification of methylated DNA from a biological sample and performing NGS to acquire DMRs that are distributed genome-wide. This method comprises (a) isolating a DNA mixture from a test sample; (b) generating an adapter-ligated DNA by ligating the DNA mixture with a sequencing adapter; (c) obtaining a MSRE-digested DNA by digesting the adapter-ligated DNA with one or more methylation-sensitive restriction endonucleases (MSREs); (d) obtaining PCR products by amplifying the MSRE-digested DNA with PCR; (e) sequencing the PCR products by next generation sequencing (NGS); and (0 determining DMRs in genome-wide scale.

In one embodiment, Method 2 further comprises the step (g) of obtaining a ratio of a chromosome copy number of the test sample to a chromosome copy number of a control sample, wherein a ratio greater than 1.34 indicates a likelihood of the presence of polysomy in the test sample.

Method 2 is for selective amplification of methylated DNA from a biological sample and performing NGS to acquire DMRs that are distributed genome-wide, which comprises (a) ligating DNA fragments with sequencing adapters, (b) digesting the adapter-ligated DNA with one or more MSREs, or a combination thereof, (c) PCR amplification of a methylated DNA fragment, (d) conducting NGS, and in the case of detecting chromosomal abnormality, (e) obtaining the ratio of reads coverage (DNA copy number) between the test chromosome and control chromosome.

In one embodiment of Method 2, the procedure includes: ligating DNA fragments with sequencing adapters, digesting the adapter ligated DNA with one or more MSREs (Table 1), employing PCR amplification to amplify methylated DNA fragments, NGS, and analyzing the sequencing data.

In one embodiment, the MSRE is AciI, BstUI, HhaI, HinPlI, HpaII or PvuI, or a combination thereof. In one embodiment, the MSRE is a combination of AciI, BstUI, HhaI, HinPlI, HpaII and PvuI. In another embodiment, the MSRE is Aor13HI, BspMII, AccIII, Aor51HI, Eco47III, BspT104104, AsuII, NspV, Eco52I, XmaIII, PluTI, PmaCI, PmlI or RsrII, or a combination thereof.

The isolation of DNA and ligation of DNA to an adapter in the method are those known in the art.

The MSRE and its embodiments are as described herein. The MSRE-digested DNAs (i.e., methylated DNA) can be obtained by digesting the adapter-ligated DNAs with one or more methylation-sensitive restriction endonucleases (MSREs). Then, the MSRE-digested DNAs are amplified by PCR.

The PCR products are sequenced by NGS. The DMRs can be determined by comparing methylated DNAs in the biological sample with those in the control sample.

NGS methods share the common feature of parallel high-throughput strategies, with the goal of lower costs in comparison to older sequencing methods. NGS methods can be broadly divided into those that typically use template amplification and those that do not. Amplification-requiring methods include pyrosequencing as commercialized by Roche as the 454 technology platforms (e.g., GS 20 and GS-FLX), the Solexa platform commercialized by Illumina, and the Supported Oligonucleotide Ligation and Detection (SOLiD) platform commercialized by Applied Biosystems. Non-amplification approaches, also known as single-molecule sequencing, are exemplified by the HeliScope platform commercialized by Helicos BioSciences, and commercialized platforms by VisiGen, Oxford Nanopore Technologies Ltd., Life Technologies/Ion Torrent, and Pacific Biosciences, respectively.

In Method 2, all sequence data are from methylated DNA. For trisomy determination, the indicator of chromosome abnormality is the ratio of chromosome copy number between test and control chromosomes from the same sample. For example, if 9.09% of DNA is fetal DNA (i.e., fetal DNA and maternal DNA are pooled as 1 versus 10), the ratio is 1.000 if the sample is normal, and the ratio tends towards 1.500 if it is from a trisomy sample. By obtaining the ratio of reads coverage between the test chromosome and control chromosome, the status of genome abnormality can be predicted. In the example, Method 2 provides improvement of 43.5% (1.500-1.045)/1.045 per site over the method based on single site qPCR. The improvement of detection power is the same as Method 1, except genome-wide screening is performed.

In another aspect, the invention provides a method (Method 3) for determining differentially methylated regions (DMRs) in genome-wide scale, comprising: (a) isolating a DNA mixture from a test sample; (b) obtaining DNA fragments by digesting the DNA mixture with one or more methylation-sensitive restriction endonucleases (MSREs); (c) generating a biotin-ligated DNA by ligating the DNA fragments with a biotin-containing linker; (d) enriching the biotin-ligated DNA with streptavidin beads; (e) obtaining an adapter-ligated DNA by ligating the enriched biotin-ligated DNA with a sequence adapter; (f) sequencing the adapter-ligated DNA by next generation sequencing (NGS); and (g) determining DMRs in genome-wide scale.

Method 3 is for selective amplification of un-methylated DNA from a mixed DNA sample and performing NGS to acquire DMRs genome-wide, which comprises (a) digesting DNA with one or more methylation sensitive enzyme or a combination thereof, (b) ligating the digested DNA with a biotin containing linker, (c) enriching the linked DNA fragment with streptavidin beads, (d) ligating the enriched DNA fragment with sequencing adapters, (e) conducting NGS to acquire DMRs genome-wide. And in the case of detecting chromosomal abnormality, it also comprises (f) analyzing the sequencing data and obtaining the ratio of reads coverage (DNA copy number) between the test chromosome and control chromosome.

In one embodiment, Method 3 further comprises the step (g) of calculating a ratio of a chromosome copy number of the test sample to a chromosome copy number of a control sample, wherein a ratio greater than 1.34 indicates a likelihood of the presence of polysomy in the test sample.

In one embodiment, the MSRE is AciI, HhaI, HinP1I, HpaII, HpyCH4IV or PvuI, or a combination thereof. In one embodiment, the MSRE is a combination of AciI, HhaI, HinPlI, HpaII, HpyCH4IV and PvuI. In another embodiment, the MSRE is Aor13HI, BspMII, AccIII, Aor51HI, Eco47III, BspT104104, AsuII, NspV, Eco52I, XmaIII, PluTI, PmaCI, PmlI or RsrII, or a combination thereof.

In one embodiment of the method of the invention, the procedure includes: digesting the DNA with a methylation sensitive enzyme; ligating the digested DNA with a biotin containing linker; enriching the linked DNA fragment with streptavidin beads; attaching the enriched DNA fragment with sequencing adapter; NGS; and analyzing the sequencing data. At this step, all sequence data are from un-methylated DNAs. The DMRs can be determined by comparing unmethylated DNAs in the biological sample with those in the control sample.

For trisomy determination, the indicator of chromosome abnormality is the ratio of read coverage between test and control chromosomes from the same sample. For example, if 9.09% of DNA is fetal DNA (i.e., fetal DNA and maternal DNA are pooled as 1 versus 10), the ratio is 1.000 if the sample is normal, and the ratio biases towards 1.500 if it is from a trisomy sample. By calculating the ratio of reads coverage between the test chromosome and control chromosome, the status of genome abnormality can be predicted. Method 3 provides improvement of 43.5% (1.500-1.045)/1.045 over the method based on single site qPCR. Method 3 enables the removal of maternal DNA that is methylated compared to unmethylated fetal DNA. Furthermore, Method 3 also enables genome wide screening.

In another aspect, the invention provides a method (Method 4) for determining differentially methylated regions (DMRs) in genome-wide scale, comprising: (a) isolating a DNA mixture from a test sample; (b) obtaining DNA fragments by digesting the DNA mixture with one or more methylation-sensitive restriction endonucleases (MSREs) wherein the unmethylated cytosines are present at the terminal nucleotides of the DNA fragments, and the methylated cytosines are present at the middle nucleotides of the DNA fragments; (c) generating a sequencing adapter-ligated DNA by ligating the DNA fragments with a sequencing adapter; (d) obtaining PCR products by amplifying the sequencing adapter-ligated DNA with PCR; (e) sequencing the PCR products by next generation sequencing (NGS); and (f) determining DMRs in genome-wide scale.

In one embodiment, Method 4 further comprises the step (g) of calculating a ratio of chromosome copy number of the test sample to the chromosome copy number of a control sample, wherein a ratio greater than 1.34 indicates a likelihood of the presence of polysomy in the test sample.

In one embodiment, the MSRE is AciI, HhaI, HinP1I, HpaII, or HpyCH4IV, or a combination thereof. In one embodiment, the MSRE is a combination of AciI, HhaI, HinP1I, HpaII and HpyCH4IV. In another embodiment, the MSRE is Aor13HI, BspMII, AccIII, Aor51HI, Eco47III, BspT104104, AsuII, NspV, Eco52I, XmaIII, PluTI, PmaCI, PmlI or RsrII, or a combination thereof.

Method 4 provides a post-sequencing identification of both methylated and unmethylated DNA, which comprises (a) digesting DNA with one or more MSRE, (b) blunting the digested DNA and adding an adenine to the 3′ end of the DNA fragment, (c) ligating the adenine protruding DNA fragment with a sequencing adapter, (d) NGS, (e) analyzing the sequencing data, wherein the cutting site with unmethylated cytosines will present at the end of the read, whereas the cutting site with methylated cytosines will present at the middle of the read. To detect chromosomal abnormality, (e) the copy number of DNA can be determined by obtaining the coverage of unmethylated reads (cutting site in the end) and methylated reads (cutting site in the middle).

In one embodiment of Method 4 of the invention, the procedure includes: digesting the DNA with MSRE, blunting the digested DNA and adding adenine to 3′ end of the DNA fragment, ligating the adenine protruding DNA fragment with a sequencing adapter; NGS; and analyzing the sequencing data. The DMRs can be determined by comparing methylated DNAs and ummethylated DNAs in the biological sample with those in the control sample.

The cutting site with unmethylated cytosines will present at the end of the read, whereas the cutting site with methylated cytosines will present at the middle of the read. At the same genomic regions, the copy number of different DNA populations can be determined by calculating the coverage of unmethylated reads (cutting site in the end) and methylated reads (cutting site in the middle). For trisomy determination, the indicator of chromosome abnormality is the ratio of read coverage between test and control chromosomes from the same sample (columns I and II in Table 7). For example, if 9.09% of DNA is fetal DNA (i.e., fetal DNA and maternal DNA are pooled as 1 versus 10), the ratio is 1.000 if the sample is normal, and the ratio tends towards 1.500 if it is from a trisomy sample. By obtaining the ratio of reads coverage between the test chromosome and control chromosome, the status of genome abnormality can be predicted. Method 4 provides improvement of 43.5% (1.500-1.045)/1.045 per site over the previous approaches based on single site qPCR. Method 3 is able to discriminate fetal DNA by detecting genome-wide MSRE cutting sites that show either hyper- or hypo-methylation compared to maternal DNA.

In another aspect, the invention provides a method (Method 5) for determining differentially methylated regions (DMRs) in genome-wide scale: (a) isolating a DNA mixture from the a test sample; (b) generating an adapter-ligated DNA by ligating the DNA mixture with a sequencing adapter; (c) obtaining a sodium bisulfite-treated DNA by treating the adapter-ligated DNA with sodium bisulfite; (d) obtaining PCR products by amplifying the sodium bisulfite-treated DNA with PCR; (e) sequencing the PCR products by next generation sequencing (NGS); and (f) determining DMRs in genome-wide scale.

In one embodiment, Method 5 further comprises the step (g) of calculating the ratio of chromosome copy number of the test sample to the chromosome copy number of a control sample, wherein a ratio greater than 1.34 indicates a likelihood of the presence of polysomy in the test sample.

Method 5 provides a post-whole Genome Bisulfite Sequencing (WGBS) identification of methylated and unmethylated DNA, which comprises (a) ligating adapters to the DNA, (b) treating the adapter-ligated DNA with sodium bisulfite, (c) PCR amplification and NGS, (d) aligning reads by separating them into two sets, one from methylated reads and another from unmethylated reads, and (e) estimating copy number from the two alignments. To detect chromosome abnormality, the following steps can be performed: (f) analyzing the alignments at DMRs to distinguish the ratio of reads from normal and abnormal chromosomes to segregate reads from different DNA; and (g) determining the genome abnormality by examining specific DMRs associated with known diseases.

Bisulfite sequencing is one of the major experimental approaches to determine the status of DNA methylation for individual cytosines. The treatment of sodium bisulfite followed by PCR converts unmethylated cytosines into thymine, whereas the methylated cytosines remain unchanged [Frommer M, McDonald L E, Millar D S, Collis C M, Watt F, Grigg G W Molloy P L, Paul C L: A genomic sequencing protocol that yields a positive display of 5-methylcytosine residues in individual DNA strands. Proc. Natl. Acad. Sci. USA 1992, 89(5): 1827-1831]. WGBS was first published in 2008 [Lister R, O'Malley R C, Tonti-Filippini J, Gregory B D, Berry C C, Millar A H, Ecker J R: Highly integrated single-base resolution maps of the epigenome in Arabidopsis. Cell 2008, 133(3):523-536] and, coupled with NGS, has become the state-of-the art method for profiling genome-wide DNA methylation at a single base resolution [Yong W S, Hsu F M, Chen P Y. Profiling genome-wide DNA methylation. Epigenetics & Chromatin, 2016, 9:26].

In one embodiment of Method 5 of the invention, the alignment of WGBS is separated into two sets: one from methylated reads and another from unmethylated reads. The copy number is estimated from the two alignments. For trisomy determination, the indicator of chromosome abnormality is the alignment with the fetal specific methylation pattern. For example, if 9.09% of DNA is fetal DNA (i.e., fetal DNA and maternal DNA are pooled as 1 versus 10), the ratio is 1.000 if the sample is normal, and the ratio tends towards 1.500 if it is from a trisomy sample. By calculating the ratio of reads coverage between the test chromosome and control chromosome, the status of genome abnormality can be predicted. Method 5 provides improvement of 43.5% (1.500-1.045)/1.045 over the method based on single site qPCR and enables genome-wide screening.

The invention could be applied to personalized medicine, by detecting genomic abnormality with significantly improved sensitivity and accuracy. For example, the invention could be applied to NIPT or cancer diagnosis.

### Applications of the Methods of the Invention

The invention is designed to discriminate DNA based on DNA methylation pattern, and is particularly useful for, but not limited to, applications that require detection of genomic variations and abnormalities, including NIPTs to detect Down syndrome and other aneuploidies, gender typing, and cancerous cell detection.

This invention could also be applied to cancer cell screening. Cancerous, also known as malignant, tumors are caused by abnormal cell proliferation. Genetic mutation, increased copy number, and changes of the DNA methylation pattern of specific genes may induce abnormal cell proliferation. Necrosis of tumor cells releases their DNA into peripheral blood, but the amount is far less compared to original blood DNA. In addition, precancerous lesions may also contain fractions of mutated DNA with genomic abnormalities. This invention has great potential to increase the proportion of DNA from tumors or lesions for cancer-screening tests, thus enhancing precision and allowing for early-stage diagnosis.

### EXAMPLES

### Example 1

### Method 1 for Enriching and Detecting Methylated DNA

Step 1. Digesting of unmethylated DNA

The DNA mixture is digested with a MSRE, such as AciI, BstUI, HhaI, HinPlI, HpaII, and PvuI, or a compatible combination thereof. The digestion reaction normally comprises 10 ng-1 μg of genomic DNA in 1×NEBuffer (NEB), and ˜1-25 U of each restriction endonuclease. The mixture is incubated at 37° C. for ˜1-12 h (depending on the enzyme) to insure complete digestion. When appropriate, the enzyme is inactivated following the protocol recommended by the manufacturer of each enzyme, and a clean-up step is performed to obtain pure digested DNA. In a preferred embodiment, non-digested DNA is directly used for fragment quantification.

### Step 2. Quantitative PCR Determination

Quantitative PCR with specific primers that target DMR regions are used to detect copy number of methylated DNA in a sample. Table 2 shows the cutoff of abnormality indicator of Method 1 of the invention.

TABLE 2

| | | | Normal | Trisomy | |
|---|---|---|---|---|---|
| | | | Sample | Sample | |
| | Proportion | | Test¹/ | Test/ | |
| | of DNA | | Control² | Control | Improvement |
| | (fetal:maternal) | | (I) | (II) | of Method 1 |
| Enrich | 1:30 | (3.23%) | 0.032 | 0.048 | 47.6% |
| methylated | 1:15 | (6.25%) | 0.063 | 0.094 | 45.5% |
| DNA followed | 1:10 | (9.09%) | 0.091 | 0.136 | 43.5% |
| by qPCR | 1:7.5 | (11.76%) | 0.118 | 0.176 | 41.6% |
| | 1:6 | (14.29%) | 0.143 | 0.214 | 40.1% |
| Without | 1:30 | (3.23%) | 1.000 | 1.016 | |
| Enrichment | 1:15 | (6.25%) | 1.000 | 1.031 | |
| (Single | 1:10 | (9.09%) | 1.000 | 1.045 | |
| site qPCR) | 1:7.5 | (11.76%) | 1.000 | 1.059 | |
| | 1:6 | (14.29%) | 1.000 | 1.071 | |

¹Test represents chromosome with putative abnormality
²Control represents normal chromosome such as chromosome 1

For example, in the trisomy determination without any DNA enrichment, if the maternal plasma contains a mixture of DNA where the mix ratio of fetal DNA and maternal DNA is 2:20, considering human chromosomes are diploid (i.e., 9.09% of the mixed DNA is fetal DNA), the ratio of chromosome copy number between test chromosome and control chromosome (e.g. chromosome 1, the largest chromosome) in a normal sample with no copy number variation is 22/22=1.000. In the case of a trisomy sample where one chromosome is triplicated, the maternal plasma contains a mixture of DNA where the mix ratio of fetal DNA and maternal DNA is 3:20 from the triplicated chromosome (e.g., chromosome 21 in Down syndrome). Therefore, the ratio of chromosome copy number between test (triplicated) and control chromosome is 23/22=1.045. The method to discriminate between trisomy and normal sample is to compare their ratios of chromosome copy number, which is 1.045/1.000=1.045, showing a very small difference of 0.045. With such a small difference, the DNA samples are difficult to distinguish when experimental noise is present.

In contrast, Method 1 provides the possibility to discriminate DNA from a mixture using differential DNA methylation patterns, with multiple novel MSREs. However, Method 1 is limited by the target sites that must show differential methylation patterns and be located at the MSRE cutting sites. The following three methods take advantage of NGS to screen genome-wide variations to examine all MSRE cutting sites, and are greatly improved over Method 1.

### Validation

The aim of our validation was to prove MSRE can significantly reduce unmethylated DNA from a DNA mix. We first amplified the test and control fragments. The test fragment contained a PmlI cutting site that is subject to MSRE digestion. The purpose was to demonstrate that a specific type of DNA can be distinguished from the DNA mix by MSRE digestion. The control fragment contains no PmlI cutting site, so no MSRE digestion occurs. The control fragment was designed to represent the original DNA mix with no enrichment of a specific type of DNA.

We methylated several DNA fragments, mixed the methylated and unmethylated fragments in a ratio of maternal bloodstream (unmethylated:methylated=10:1), digested the fragment with MSRE, and quantified the methylated DNA by qPCR.

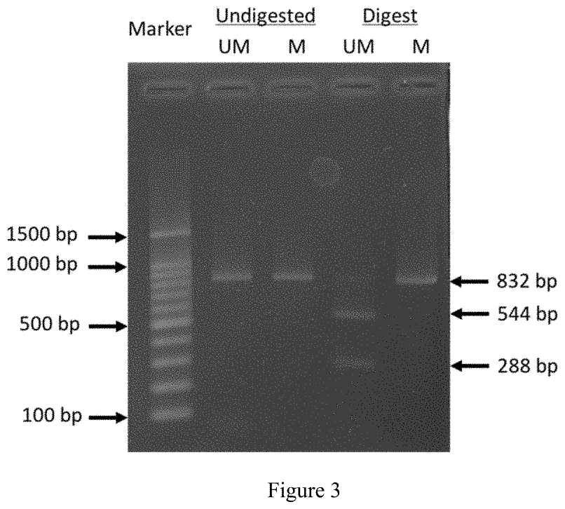

The novel MSRE PmlI was selected for validation. We amplified a 832-bp DNA fragment that contains one PmlI cutting site (test fragment) using PCR. The PCR product was free of DNA methylation, and was aliquoted into two tubes. We methylated the DNA in one tube using SssI methyltransferase while the DNA in the other tube remained unmethylated. FIG. 3 refers to the electrophoresis result of PCR products undigested or digested by novel MSRE PmlI. As expected, PmlI digested unmethylated DNA into 2 fragments (544 bp and 288 bp), whereas methylated DNA sequences were not digested, suggesting that DNA can be effectively distinguished by methylation status using PmlI digestion.

The 832-bp test fragment was amplified and one aliquot was methylated. The unmethylated fragment was digested whereas the methylated fragment remained intact, suggesting there is one PmlI cutting site in the test fragment.

To distinguish between maternal and fetal DNA in the maternal bloodstream in which the maternal DNA:fetal DNA is 10:1 (no aneuploidy), we pooled 11 portions of fragments, i.e., 10 portions of unmethylated test fragment and 1 portion of methylated test fragment (maternal:fetal =10:1). To mimic the aneuploidy condition, we pooled 11.5 portions of fragments: 1.5 portion of methylated test fragment and 10 portions of unmethylated test fragment. The test fragment was an 832 bp PCR product with one PmlI cutting site, and the control fragment was a 536 bp PCR product with no PmlI cutting site. We added 11 portions of control fragment to all test samples. Since there was no PmlI site inside the control fragment, it should not be digested even without methylation. The pooled DNA, simulating both conditions, was divided into two tubes. One was subjected to digestion using the test enzyme and the other was left undigested. The latter was diluted and used for qPCR as template. Since the control fragment had no PmlI cutting site, the ratio of unmethylated and methylated control fragments should not be affected by PmlI digestion. Primers were designed for qPCR to quantify the digested and undigested groups of both normal and aneuploidy condition.

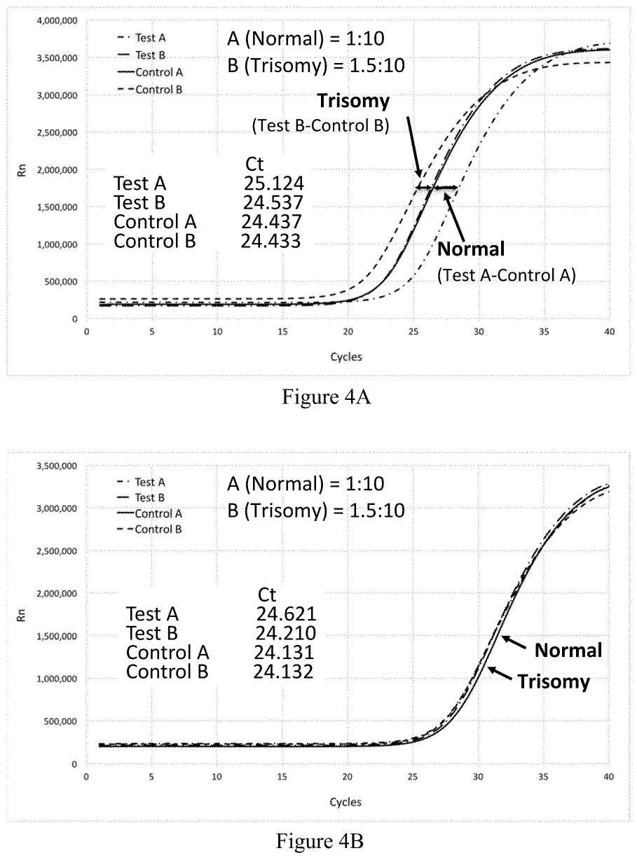

The amplification plots of the digested fragment of normal versus trisomy condition and the undigested fragments are shown in FIG. 4A and 4B, respectively). We found a clear difference in the cycle of threshold (Ct) values of digested test fragments in the normal and aneuploid condition (FIG. 4A), whereas the undigested fragments showed no difference (FIG. 4B). Table 3 shows a summary of the validation result. For each sample, we calculated the relative concentration of test DNA (1/2\hat{ }Cₜₑₛₜ-C_{control}). The digested method showed 1.498-fold changes between trisomy and normal sample (Test/Control), whereas the undigested method showed 1.32-fold changes between trisomy and normal sample. In theory, the relative ratio between trisomy and normal is 1.5 (1.5/1.0), and from our result, 1.498 from the method with digestion was closer to 1.5, rather than the 1.32 from the method without digestion. Since other polysomies (tetrasomy, pentasomy, hexasomy and so forth) have more than three copies of the chromosomes, the relative ratio between polysomy and normal is expected to be a value more than 1.498.

The method with PmlI digestion showed 13.5% [(1.498-1.32)/1.32] improved accuracy. This confirms that PmlI is a novel MSRE that can enrich fetal DNA (methylated DNA) from a maternal/fetal mix of DNA and therefore further enhances the test accuracy of the method.

TABLE 3

| | With digestion | | | | Without digestion | | | |
|---|---|---|---|---|---|---|---|---|
| | Sample | | | | | | | |
| | Normal | | Trisomy | | Normal | | Trisomy | |
| qPCR primers | Control | Test | Control | Test | Control | Test | Control | Test |
| Ct value | 24.437 | 25.124 | 24.433 | 24.537 | 24.131 | 24.621 | 24.132 | 24.210 |

| | | | | |
|---|---|---|---|---|
| Relative Test | 0.621 | 0.930 | 0.712 | 0.940 |
| Conc. (Test/Control) | | | | |

| | | |
|---|---|---|
| Ratio to | 1.498 | 1.320 |
| discriminate | | |
| between trisomy | | |
| and normal sample | | |

### Example 2

### Method 2 for Selective Amplification of Methylated DNA

Step 1. Adapter Ligation

Isolated DNA has at least three types of ends: 3′ overhangs, 5′ overhangs, and blunt ends. In order to ligate the adapters (Illumina, Inc.) to target DNA, the ends of DNA fragments need to be repaired. Purified cell-free DNA fragments are first end filled-in by T4 DNA polymerase in the presence of 40 μM dNTP, then addition of 5′-phosphates to oligonucleotide and removal of 3′-phosphoryl groups are performed by T4 Polynucleotide Kinase, followed by treatment with Klenow Fragment DNA polymerase (3→5′ exo-) in the presence of 200 μM dATP to generate 3′-end adenine DNA fragments. Double-stranded adapter oligonucleotides are then ligated to both 5′ and 3′ ends of end-repaired and a-tailing DNA. These oligonucleotides can be designed according to different sequencing platforms.

### Step 2. Digesting of Unmethylated DNA

The DNA mixture is digested with one or more MSREs, such as AciI, BstUI, HhaI, HinPlI, HpaII, and PvuI, or a compatible combination thereof. The digestion reaction normally comprises from 10 ng-1 μg of genomic DNA in 1×NEBuffer (NEB), and ˜1-25 U of each restriction endonuclease. The mixture is incubated at 37° C. for ˜1-12 h (depending on the enzyme) to insure complete digestion. When the digestion is completed, the enzyme is inactivated following the protocol recommended by the manufacturer of each enzyme, and a clean-up step is performed to obtain pure digested DNA. In a preferred embodiment, the non-digested DNA sample is directly used for PCR enrichment.

### Step 3. PCR Enrichment and NGS

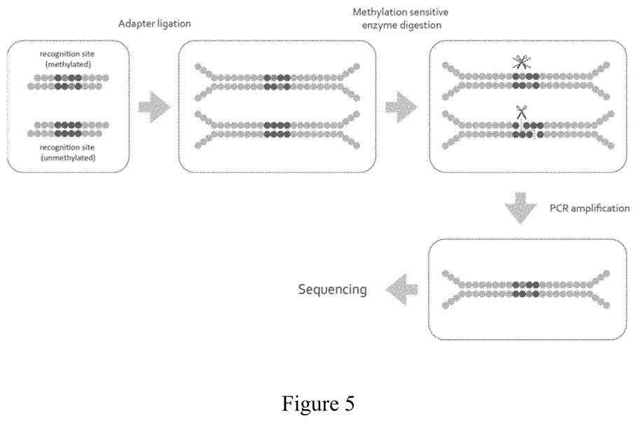

Primers specific for the adapters are used to amplify the methylated DNA. The amplified DNA is then sequenced, as shown in FIG. 5. Table 4 shows the cutoff of abnormality indicator of Method 2 of the invention with and without DNA enrichment.

TABLE 4

| | | | Normal | Trisomy | |
|---|---|---|---|---|---|
| | | | Sample | Sample | |
| | Proportion | | Test¹/ | Test/ | |
| | of DNA | | Control² | Control | Improvement |
| | (fetal:maternal) | | (I) | (II) | of Method 2 |
| Method 2: | 1:30 | (3.23%) | 1.000 | 1.500 | 47.6% |
| Enriching | 1:15 | (6.25%) | 1.000 | 1.500 | 45.5% |
| methylated | 1:10 | (9.09%) | 1.000 | 1.500 | 43.5% |
| DNA followed | 1:7.5 | (11.76%) | 1.000 | 1.500 | 41.6% |
| by NGS | 1:6 | (14.29%) | 1.000 | 1.500 | 40.1% |
| Without | 1:30 | (3.23%) | 1.000 | 1.016 | |
| Enrichment | 1:15 | (6.25%) | 1.000 | 1.031 | |
| (Single | 1:10 | (9.09%) | 1.000 | 1.045 | |
| site qPCR) | 1:7.5 | (11.76%) | 1.000 | 1.059 | |
| | 1:6 | (14.29%) | 1.000 | 1.071 | |

¹Test represents reads from chromosome with putative abnormality
²Control represents reads from normal chromosome such as chromosome 1

Method 2 provides the possibility to enrich methylated DNA from a mixture by differential DNA methylation patterns.

### Validation

The aim of our validation was to prove Method 2 can significantly reduce unmethylated DNA from a DNA mixture using NGS technology. We first amplified the test fragment containing a PvuI cutting site that is subject to MSRE digestion. The purpose was to show a specific type of DNA can be distinguished from a DNA mix by MSRE digestion.

The PCR product of the test fragment was free of DNA methylation, and was aliquoted into two tubes. We methylated the DNA in one tube using SssI methyltransferase and the DNA in the other tube remained unmethylated. We then mixed the methylated and unmethylated fragments at a ratio of 1:1, generated the NGS library (including end repair, A-tailing, and ligation of the fragments with sequencing adapters), digested the library DNA with MSRE, and quantified the methylated and unmethylated DNA.

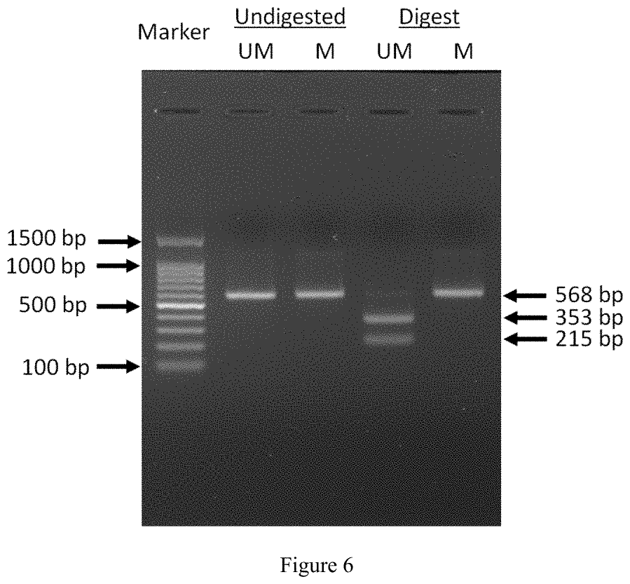

To distinguish methylated and unmethylated DNA, we used barcoded primers to label the methylated and unmethylated DNA. We amplified a 568 bp DNA fragment that contained one PvuI cutting site (test fragment) using PCR. The PCR product was free of DNA methylation. The methylated test DNA was generated using SssI methyltransferase. The results of the digestion are shown in FIG. 6. As expected, PvuI digested the unmethylated DNA into 2 fragments (353 bp and 215 bp), whereas methylated DNA sequences were not digested, suggesting that DNAs can be effectively distinguished by their methylation status using PvuI digestion.

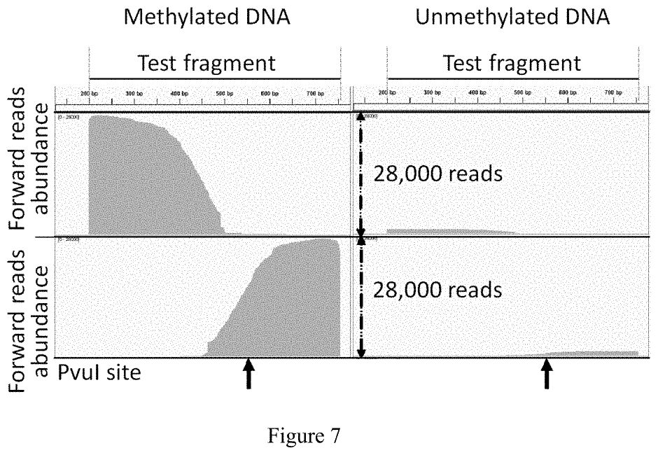

The pooled DNA that contained methylated and unmethylated test DNA was used to generate as NGS library using standard protocols. After library construction, the library DNA was than treated with PvuI to digest the unmethylated DNA fragments. After PCR amplification, the DNA was then sequenced using NGS. Reads were mapped using Bowtie 2 and the number of reads for methylated and unmethylated DNA fragments was calculated. We yielded 28,524 fragments from the NGS; 27,395 were methylated DNA and 1,129 were unmethylated DNA fragments, a ratio of 33.12:1 (FIG. 7). In theory, we expected no unmethylated DNA fragments (0%), but we obtained 2.9% of reads from unmethylated DNA. This less than expected improvement may have been due to MSRE efficiency. However, our results confirm that our method can enrich methylated DNA from a mix of DNAs, since the ratio of methylated DNA and unmethylated DNA was reduced from 1 to 1,129/27,395=0.04.

### Example 3

### Method 3 for Selective Amplification of Unmethylated DNA

### Step 1. Digestion of Unmethylated DNA

DNAs were digested with one or more MSREs, such as AciI, HhaI, HinP1I, HpaII, HpyCH4IV, and PvuI to produce either 5′ overhangs or 3′ overhangs. The digestion reaction normally comprises 10 ng-1 μg of genomic DNA in 1×NEBuffer (NEB), and ˜1-25 U of each restriction endonuclease. The mixture is incubated at 37° C. for ˜1 to 12 hours (depending on the enzyme) to insure complete digestion. When the digestion is completed, the enzyme is inactivated Step 2. Linker ligation

The following ligation procedure is designed to work with DNA that has been digested with restriction enzyme, resulting in ends with either 5′ overhang, or 3′ overhang. The structure of the linker is based on the type of ends generated by the restriction endonuclease. The linker is composed of two oligonucleotides, which are hybridized to each other at regions along their length. The length of the short oligonucleotide is about 7 bp to about 15 bp, with biotin at 5′ end. The structure of the linker is developed to minimize the ligation of linker to each other by the presence of about a 5 bp 5′ overhang that prevents ligation in the opposite orientation. A typical ligation procedure involves the incubation of about 1 to about 100 ng of DNA in 1×T4 DNA ligase buffer, about 10- about 100 pmol of each linker, and about 400- about 2,000 Units of T4 DNA Ligase. Ligations are performed at 25° C. for 1 hour, followed by inactivation of the ligase at 75° C. for 15 minutes.

### Step 3. Biotinylated DNA Fragment Enrichment

Ligation products were mixed with 100 μg M-280 dynabeads and incubated at room temperature for 30 minutes. After incubation, the beads were washed 4 times with 70 μl of TE buffer, 2 times with 70 μl of freshly prepared 0.1 N KOH, and 4 times with 80 μl of TE buffer. To dissociate biotinylated nucleic acids from Streptavidin-beads, the beads were incubated in 95% formamide+10 mM EDTA, pH 8.2 for 5 minutes at 65° C.

### Step 4. Adapter Ligation

DNA fragments were end filled-in by T4 DNA polymerase, followed by Klenow DNA polymerase (exo-) to generate 3′-end adenine DNA fragments. Double stranded adaptor oligonucleotides were ligated to both 5′ and 3′ ends of end-repaired DNA. These oligonucleotides could be designed according to different sequencing platforms.

### Step 5. PCR Enrichment and Next Generation Sequencing

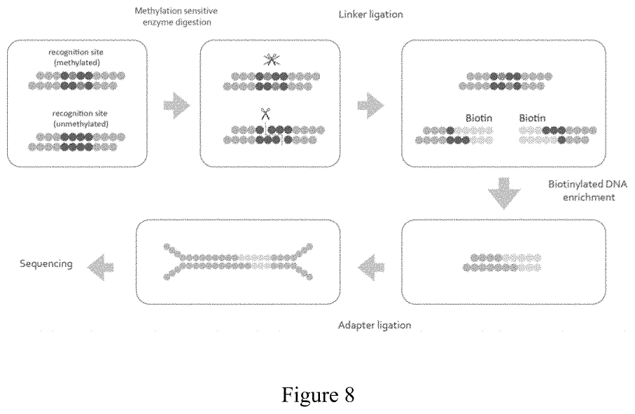

Primers specific for adaptor were used to amplify the methylated DNA. The amplified DNA was then sequenced, as shown in FIG. 8. Table 5 shows the cutoff of abnormality indicator with Method 3 with and without DNA enrichment.

TABLE 5

| | | | Normal | Trisomy | |
|---|---|---|---|---|---|
| | | | Sample | Sample | |
| | Proportion | | Test¹/ | Test/ | |
| | of DNA | | Control² | Control | Improvement |
| | (fetal:materal) | | (I) | (II) | of Method 3 |
| Method 3: | 1:30 | (3.23%) | 1.000 | 1.500 | 47.6% |
| Enriching | 1:15 | (6.25%) | 1.000 | 1.500 | 45.5% |
| un-methylated | 1:10 | (9.09%) | 1.000 | 1.500 | 43.5% |
| DNA followed | 1:7.5 | (11.76%) | 1.000 | 1.500 | 41.6% |
| by NGS | 1:6 | (14.29%) | 1.000 | 1.500 | 40.1% |
| Without | 1:30 | (3.23%) | 1.000 | 1.016 | |
| Enrichment | 1:15 | (6.25%) | 1.000 | 1.031 | |
| (Single | 1:10 | (9.09%) | 1.000 | 1.045 | |
| site qPCR) | 1:7.5 | (11.76%) | 1.000 | 1.059 | |
| | 1:6 | (14.29%) | 1.000 | 1.071 | |

¹Test represents reads from chromosome with putative abnormality
²Control represents reads from normal chromosome such as chromosome 1

### Example 4

### Method 4 for Selective Amplification of Un-methylated DNA

### Step 1. Digesting of Un-methylated DNA

DNA is digested with an MSRE, such as Acil, HhaI, HinP1I, HpaII, and HpyCH4IV to produce either 5′ overhang or 3′ overhang. The digestion reaction usually comprises from 10 ng to 1 μg of genomic DNA in 25-100/μl of 1×NEBuffer (NEB), and about 1 to about 25 units of each restriction endonuclease. The mixture is incubated at 37° C. for 2 h to insure complete digestion. When appropriate, the enzyme is inactivated at 65 ° C. for 15 minutes and the sample is precipitated and resuspended to a final concentration of 1 to 50 ng/μl.

### Step 2. Adapter Ligation

In order to ligate the adapters to target DNA, the ends of the DNA fragments need to be repaired. DNA fragments are first end filled-in by T4 DNA polymerase in the presence of 40 μM dNTP, 5′-phosphates are added and 3′-phosphoryl groups are removed from oligonucleotides by T4 Polynucleotide Kinase, followed by treatment with Klenow Fragment DNA polymerase (3′→5′ exo-) in the presence of 200 μM dATP to generate 3′-end adenine DNA fragments. Double-stranded adapter oligonucleotides are then ligated to both 5′ and 3′ ends of end-repaired and a-tailing DNA. These oligonucleotides can be designed according to different sequencing platforms.

### Step 3. PCR Enrichment and Next Generation Sequencing

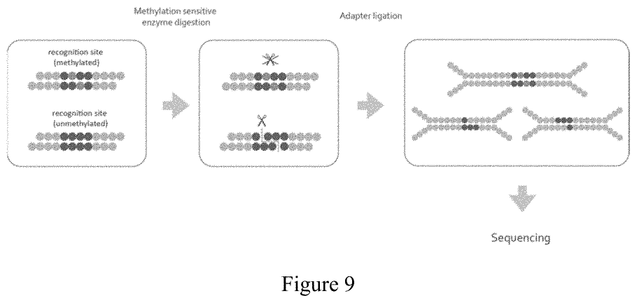

Primers specific for adapters are used to amplify the DNA library. The amplified DNA is then sequenced as shown in FIG. 9. Table 6 shows the cutoff of abnormality indicator of Method 4 of the invention with and without DNA enrichment.

TABLE 6

| | | | Normal | Trisomy | |
|---|---|---|---|---|---|
| | | | Sample | Sample | |
| | Proportion | | Test¹/ | Test/ | |
| | of DNA | | Control² | Control | Improvement |
| | (fetal:maternal) | | (I) | (II) | of Method 4 |
| Method 4: | 1:30 | (3.23%) | 1.000 | 1.500 | 47.6% |
| Post-sequencing | 1:15 | (6.25%) | 1.000 | 1.500 | 45.5% |
| identification | 1:10 | (9.09%) | 1.000 | 1.500 | 43.5% |
| of methylated and | 1:7.5 | (11.76%) | 1.000 | 1.500 | 41.6% |
| unmethylated | 1:6 | (14.29%) | 1.000 | 1.500 | 40.1% |
| DNA with NGS | | | | | |
| Without | 1:30 | (3.23%) | 1.000 | 1.016 | |
| Enrichment | 1:15 | (6.25%) | 1.000 | 1.031 | |
| (Single | 1:10 | (9.09%) | 1.000 | 1.045 | |
| site qPCR) | 1:7.5 | (11.76%) | 1.000 | 1.059 | |
| | 1:6 | (14.29%) | 1.000 | 1.071 | |

¹Test represents reads from chromosome with putative abnormality
²Control represents reads from normal chromosome such as chromosome 1

Method 4 provides a post-NGS identification method to differentiate methylated and unmethylated DNA from a DNA mixture by differential methylation patterns. Validation

The aim of our validation was to show Method 4 can differentiate methylated and unmethylated DNA from a DNA mixture using NGS technology. We first amplified the test fragment containing a PvuI cutting site that is subject to MSRE digestion. The purpose was to show a specific type of DNA can be distinguished from the DNA mix by MSRE digestion.

The PCR product was free of DNA methylation, and was aliquoted into two tubes. We methylated the DNA in one tube using SssI methyltransferase and the DNA in the other tube remained unmethylated. We mixed the methylated and unmethylated fragments at a ratio of 1:1, then digested the DNA with MSRE and purified the undigested methylated DNA and digested unmethylated DNA. These purified DNA fragments were used to generate libraries for NGS (including end repair, A-tailing and ligation of the fragments with sequencing adapters).

To distinguish between methylated and unmethylated DNA using NGS, we used barcoded primers to label the methylated and unmethylated DNA. We amplified a 568 bp DNA fragment that contains one PvuI cutting site (test fragment) using PCR. The PCR product was free of DNA methylation. The methylated test DNA was generated using SssI methyltransferase. The digestion result is shown in FIG. 6. As expected, PvuI digested unmethylated DNA into 2 fragments (353 bp and 215 bp), whereas methylated DNA sequences were not digested, suggesting that the DNAs can be effectively distinguished by their methylation status using PvuI digestion.

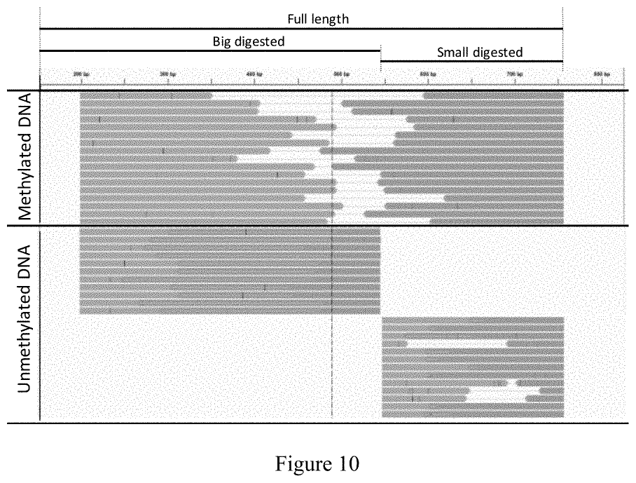

The pooled DNA that contains methylated and unmethylated test DNA was first treated with PvuI, which digests unmethylated DNA. The enzyme-treated DNA was then used for generating an NGS library using standard protocols. After PCR amplification, the DNA library was then sequenced using NGS. Reads were separated into methylated and unmethylated by the attached barcodes and were mapped to the reference using Bowtie 2. Reads of undigested DNA (568 bp, full length) would contain PvuI sites and reads of digested DNA (353 bp and 215 bp) would map to the same location without PvuI sites in the alignments. For the methylated DNA library, we yielded 6,208 fragment from NGS, in which 6135 were full length and 73 were digested fragments (FIG. 10; Table 7). In theory, we expected no digested DNA fragments from methylated DNA, while we obtained 98% of reads that were full-length DNA fragments. This less than expected improvement may have been due to methylation efficiency. For the unmethylated DNA, we yielded 4,703 fragments, in which 190 were full length and 4,513 (353 bp: 4,439 +215 bp: 74) were digested fragments (Table 6). In theory, we expected all DNA fragments from unmethylated DNA to be digested, while 96% of the reads were digested DNA fragments. However, our results confirm that Method 4 can distinguish methylated and unmethylated DNA from a mix of DNAs. Table 7 shows statistical analysis of Method 4 validation.

TABLE 7

| | # full | | # small | |
|---|---|---|---|---|
| | length | # digested | digested | Ratio (# full |
| | fragment | fragment | fragment | length:# |
| Sample | (568 bp) | (353) | (215) | digested) |
| Methylated DNA | 6,135 | 73 | 0 | 98.82 |
| Unmethylated DNA | 190 | 4,439 | 74 | 0.04 |

### Example 5

### Method 5 for Selective Amplification of Unmethylated DNA

### Step 1. Adapter Ligation

The reaction normally comprises 10 ng-1 μg of genomic DNA. DNA fragments are first end-repaired by T4 DNA polymerase in the presence of 40 μM dNTP; 5′-phosphates are added and of 3′-phosphoryl groups are removed from oligonucleotides by T4 Polynucleotide Kinase, followed by treatment with Klenow Fragment DNA polymerase (3′→5′ exo-) in the presence of 200 μM dATP to generate 3′-end adenine DNA fragments. Double stranded adapter oligonucleotides are then ligated to both 5′ and 3′ ends of end-repaired and a-tailing DNA. These oligonucleotides can be designed according to different sequencing platforms.

### Step 2. Bisulfite Conversion

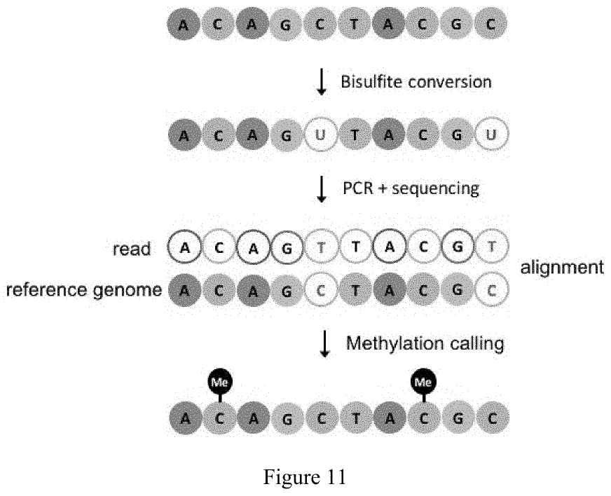

Adapter-ligated DNA is treated with sodium bisulfate (see FIG. 11) according the manufacture's protocol (Qiagen EpiTect Fast Bisulfite Conversion kit).

### Step 3. PCR Enrichment and Next Generation Sequencing

Primers specific for adapters are used to amplify the DNA library. The DNA library is then sequenced. Table 8 shows the cutoff of abnormality indicator with Method 5 of the invention with and without DNA enrichment.

TABLE 8

| | | | Normal | Trisomy | |
|---|---|---|---|---|---|
| | | | Sample | Sample | |
| | Proportion | | Test¹/ | Test/ | |
| | of DNA | | Control² | Control | Improvement |
| | (fetal:maternal) | | (I) | (II) | of Method 5 |
| Method 5: | 1:30 | (3.23%) | 1.000 | 1.500 | 47.6% |
| Post-WGBS | 1:15 | (6.25%) | 1.000 | 1.500 | 45.5% |
| identification | 1:10 | (9.09%) | 1.000 | 1.500 | 43.5% |
| of methylated | 1:7.5 | (11.76%) | 1.000 | 1.500 | 41.6% |
| and unmethylated | 1:6 | (14.29%) | 1.000 | 1.500 | 40.1% |
| DNA | | | | | |
| Without | 1:30 | (3.23%) | 1.000 | 1.016 | |
| Enrichment | 1:15 | (6.25%) | 1.000 | 1.031 | |
| (Single | 1:10 | (9.09%) | 1.000 | 1.045 | |
| site qPCR) | 1:7.5 | (11.76%) | 1.000 | 1.059 | |
| | 1:6 | (14.29%) | 1.000 | 1.071 | |

¹Test represents reads from chromosome with putative abnormality
²Control represents reads from normal chromosome such as chromosome 1

Method 5 provides a possibility to differentiate methylated and unmethylated DNA from a DNA mixture by differential DNA methylation patterns.

### Validation

The aim of our validation was to prove our Method 5 can differentiate methylated and unmethylated DNA from a DNA mixture using WGBS technology. We first amplified the test fragment containing a CpG site that is subject to methylation. The purpose was to show a specific type of DNA can be distinguished from the DNA mix by bisulfite conversion. Preparation of reduced representation bisulfite sequencing libraries for genome-scale DNA methylation profiling was previously described in Nature Protocols. Volume: 6, Pages: 468-481.

The PCR product was free of DNA methylation, and was aliquoted into two tubes. We methylated the DNA in one tube using SssI methyltransferase and the DNA in the other tube remained unmethylated. We mixed the methylated and unmethylated fragment at a ratio of 1:1, generated the WGBS library, and then quantified methylated and unmethylated reads.

To distinguish between methylated and unmethylated DNA using NGS, we used barcoded primers to label the methylated and unmethylated DNA. We amplified a 636 bp DNA fragment that contains CpG sites (test fragment) using PCR amplification. The PCR product was free of DNA methylation. The methylated test DNA was generated using SssI methyltransferase.

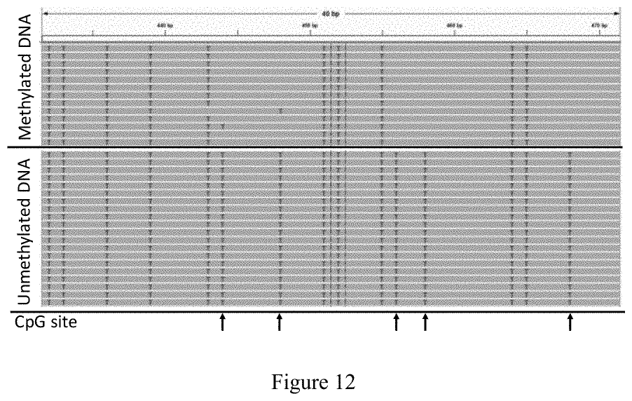

The pooled DNA that contained methylated and unmethylated test DNA was used for generating a WGBS library using standard protocols. After PCR amplification, the DNA was then sequenced using NGS. Reads were separated into methylated and unmethylated by the attached barcodes and mapped to the reference genome using BS-seeker 2, which is designed for bisulfate sequencing. For the methylated DNA, we yielded 435 fragments, in which 398 were methylated (C) and 37 were unmethylated (T) (FIG. 12; Table 9). In theory, we expected all DNA fragments to be methylated; however, 91% of our reads were methylated. This less than expected improvement may have been due to methylation efficiency. For the unmethylated DNA, we yielded 1,399 fragments from NGS in which all 1,399 fragments were unmethylated (T) (Table 9). We obtained 100% of unmethylated reads as expected. This confirms that Method 5 can distinguish methylated and unmethylated DNA from a mix of DNAs. Table 9 shows statistical analysis of Method 5 validation.

TABLE 9

| | # Methylated | # Unmethylated |
|---|---|---|
| | fragments | fragments |
| Sample | from BS-seq (C) | from BS-seq (T) |
| Methylated DNA | 398 | 37 |
| Unmethylated DNA | 0 | 1,399 |

1. A method for detecting differentially methylated regions (DMR) comprising using one or more methylation-sensitive restriction endonucleases (MSREs) selected from the group consisting of Aor13HI, BspMII, AccIII, Aor51HI, Eco47III, BspT104104, AsuII, NspV, Eco52I, XmaIII, PluTI, PmaCI, PmlI and RsrII.

2. A method for detecting polysomy in a test sample comprising fetal DNAs and maternal DNAs, comprising:
(a) isolating a DNA mixture from the test sample and a control sample;
(b) obtaining DNA fragments by digesting the DNA mixture with one or more methylation-sensitive restriction endonucleases (MSREs);
(c) amplifying specific differentially methylated regions (DMRs) by subjecting the DNA fragments to PCR amplification; and
(d) obtaining a ratio of the relative concentration of methylated fetal DNAs in the test sample to the relative concentration of methylated fetal DNAs in the control sample,
wherein the relative concentration of methylated fetal DNAs in the test sample greater than that of the control sample indicates a likelihood of the presence of the polysomy in the test sample.

3. The method of claim 2, wherein the polysomy is trisomy.

2. The method of claim 2, wherein the ratio greater than 1.34 indicates a likelihood of the presence of the polysomy in the test sample.

5. The method of claim 2, wherein the ratio greater than 1.49 indicates a likelihood of the presence of the polysomy in the test sample.

6. The method of claim 2, wherein when the ratio of the concentration of ratio of copy number of fetal DNA to total copy number of the DNA mixture is less than 10%, the method shows at least 13.5% improvement as compared to a method without the step of digestion.

7. The method of claim 2, wherein when the ratio of the concentration of ratio of copy number of fetal DNA to total copy number of the DNA mixture is less than 15%, the method shows at least 40% improvement as compared to a method without the step of digestion.

8. The method of claim 2, wherein the MSRE is selected from the group consisting of AatlI, AccII, FnuDII, AciI, AclI, AfeI, AgeI, Aor13HI, BspMII, AccIII, Aor51HI, Eco47III, AscI, AsiSI, AvaI, BceAI, BmgBI, BsaAI, BsaHI, BsiEI, BsiWI, BsmBI, BspDI, BspT104104, AsuII, NspV, BsrFI, BssHII, BstBI, BstUI, Cfr10I, ClaI, EagI, Eco52I, XmaIII, FauI, FseI, FspI, HaeII, HgaI, HhaI, HinP1I, HpaII, Hpy99I, HpyCH4IV, KasI, MluI, Nael, NarI, NgoMIV, NotI, NruI, PaeR7I, PluTI, PmaCI, PmlI, PvuI, RsrII, SacII, SalI, SfoI, SgrAI, SmaI, SnaBI, TspMI and ZraI.

9. The method of claim 8, wherein the MSRE is selected from the group consisting of Aor13HI, BspMII, AccIII, Aor51HI, Eco47III, BspT104104, AsuII, NspV, Eco52I, XmaIII, PluTI, PmaCI, PmlI and RsrII.

10. A method for determining differentially methylated regions (DMRs) in genome-wide scale, comprising:
(a) isolating a DNA mixture from a test sample;
(b) generating an adapter-ligated DNA by ligating the DNA mixture with a sequencing adapter;
(c) obtaining a MSRE-digested DNA by digesting the adapter-ligated DNA with one or more methylation-sensitive restriction endonucleases (MSREs);
(d) obtaining PCR products by amplifying the MSRE-digested DNA with PCR;
(e) sequencing the PCR products by next generation sequencing (NGS); and
(f) determining DMRs in genome-wide scale .

11. The method of claim 10, further comprising the step of calculating the ratio of chromosome copy number of the test sample to the chromosome copy number of a control sample, wherein a ratio greater than 1.34 indicates a likelihood of the presence of polysomy in the test sample.

12. The method of claim 11, wherein the polysomy is trisomy.

13. The method of claim 10, wherein the MSRE is selected from the group consisting of AatlI, AccII, FnuDII, AciI, AclI, AfeI, AgeI, Aor13HI, BspMII, AccIII, Aor51HI, Eco47III, AscI, AsiSI, AvaI, BceAI, BmgBI, BsaAI, BsaHI, BsiEI, BsiWI, BsmBI, BspDI, BspT104104, AsuII, NspV, BsrFI, BssHII, BstBI, BstUI, Cfr10I, ClaI, EagI, Eco52I, XmaIII, Faul, Fsel, FspI, HaeII, HgaI, HhaI, HinP1I, HpaII, Hpy99I, HpyCH4IV, KasI, MluI, Nael, NarI, NgoMIV, NotI, NruI, PaeR7I, PluTI, PmaCI, PmlI, PvuI, RsrII, SacII, SalI, SfoI, SgrAI, SmaI, SnaBI, TspMI and ZraI.

14. The method of claim 13, wherein the MSRE is selected from the group consisting of Aor13HI, BspMII, AccIII, Aor51HI, Eco47III, BspT104104, AsuII, NspV, Eco52I, XmaIII, PluTI, PmaCI, PmlI and RsrII.

15. A method for determining differentially methylated regions (DMRs) in genome-wide scale, comprising:
(a) isolating a DNA mixture from a test sample;
(b) obtaining DNA fragments by digesting the DNA mixture with one or more methylation-sensitive restriction endonucleases (MSREs);
(c) generating a biotin-ligated DNA by ligating the DNA fragments with a biotin-containing linker;
(d) enriching the biotin-ligated DNA with streptavidin beads;
(e) obtaining an adapter-ligated DNA by ligating the enriched biotin-ligated DNA with a sequence adapter;
(f) sequencing the adapter-ligated DNA by next generation sequencing (NGS); and
(g) determining DMRs in genome-wide scale.

16. The method of claim 15, further comprising the step of calculating the ratio of chromosome copy number of the test sample to the chromosome copy number of a control sample, wherein a ratio greater than 1.34 indicates a likelihood of the presence of polysomy in the test sample.

17. The method of claim 16, wherein the polysomy is trisomy.

18. The method of claim 15, wherein the MSRE is selected from the group consisting of AatlI, AccII, FnuDII, AciI, AclI, AfeI, AgeI, Aor13HI, BspMII, AccIII, Aor51HI, Eco47III, AscI, AsiSI, AvaI, BceAI, BmgBI, BsaAI, BsaHI, BsiEI, BsiWI, BsmBI, BspDI, BspT104104, AsuII, NspV, BsrFI, BssHII, BstBI, BstUI, Cfr10I, ClaI, EagI, Eco52I, XmaIII, FauI, FseI, FspI, HaeII, HgaI, HhaI, HinPlI, HpaII, Hpy99I, HpyCH4IV, KasI, MluI, NaeI, NarI, NgoMIV, NotI, NruI, PaeR7I, PluTI, PmaCI, PmlI, PvuI, RsrII, SacII, SalI, SfoI, SgrAI, SmaI, SnaBI, TspMI and ZraI.

19. The method of claim 18, wherein the MSRE is selected from the group consisting of Aor13HI, BspMII, AccIII, Aor51HI, Eco47III, BspT104104, AsuII, NspV, Eco52I, XmaIII, PluTI, PmaCI, PmlI and RsrII.

20. A method for determining differentially methylated regions (DMRs) in genome-wide scale, comprising:
(a) isolating a DNA mixture from a test sample;
(b) obtaining DNA fragments by digesting the DNA mixture with one or more methylation-sensitive restriction endonucleases (MSREs) wherein the unmethylated cytosines are present at the terminal nucleotides of the DNA fragments, and the methylated cytosines are present at the middle nucleotides of the DNA fragments;
(c) generating a sequencing adapter-ligated DNA by ligating the DNA fragments with a sequencing adapter;
(d) obtaining PCR products by amplifying the sequencing adapter-ligated DNA with PCR;
(e) sequencing the PCR products by next generation sequencing (NGS); and
(f) determining DMRs in genome-wide scale.

21. The method of claim 20, further comprising the step of calculating the ratio of chromosome copy number of the test sample to the chromosome copy number of a control sample, wherein a ratio greater than 1.34 indicates a likelihood of the presence of polysomy in the test sample.

22. The method of claim 21, wherein the polysomy is trisomy.

23. The method of claim 20, wherein the MSRE is selected from the group consisting of AatlI, AccII, FnuDII, AciI, AclI, AfeI, AgeI, Aor13HI, BspMII, AccIII, Aor51HI, Eco47III, AscI, AsiSI, AvaI, BceAI, BmgBI, BsaAI, BsaHI, BsiEI, BsiWI, BsmBI, BspDI, BspT104104, AsuII, NspV, BsrFI, BssHII, BstBI, BstUI, Cfr10I, ClaI, EagI, Eco52I, XmaIII, FauI, FseI, FspI, HaeII, HgaI, HhaI, HinPlI, HpaII, Hpy99I, HpyCH4IV, KasI, MluI, NaeI, NarI, NgoMIV, NofI, NruI, PaeR7I, PluTI, PmaCI, PmlI, PvuI, RsrII, SacII, SalI, SfoI, SgrAI, SmaI, SnaBI, TspMI and ZraI.

24. The method of claim 23, wherein the MSRE is selected from the group consisting of Aor13HI, BspMII, AccIII, Aor51HI, Eco47III, BspT104104, AsuII, NspV, Eco52I, XmaIII, PluTI, PmaCI, PmlI and RsrII.

25. A method for determining differentially methylated regions (DMRs) in genome-wide scale:
(a) isolating a DNA mixture from a test sample;
(b) generating an adapter-ligated DNA by ligating the DNA mixture with a sequencing adapter;
(c) obtaining a sodium bisulfite-treated DNA by treating the adapter-ligated DNA with sodium bisulfite;
(d) obtaining PCR products by amplifying the sodium bisulfite-treated DNA with PCR;
(e) sequencing the PCR products by next generation sequencing (NGS); and
(f) determining DMRs in genome-wide scale.

26. The method of claim 25, further comprising the step of calculating the ratio of chromosome copy number of the test sample to the chromosome copy number of a control sample, wherein a ratio greater than 1.34 indicates a likelihood of the presence of polysomy in the test sample.

27. The method of claim 26, wherein the polysomy is trisomy.

28. The method of claim 25, wherein the MSRE is selected from the group consisting of AatlI, AccII, FnuDII, AciI, AclI, AfeI, AgeI, Aor13HI, BspMII, AccIII, Aor51HI, Eco47III, AscI, AsiSI, AvaI, BceAI, BmgBI, BsaAI, BsaHI, BsiEI, BsiWI, BsmBI, BspDI, BspT104104, AsuII, NspV, BsrFI, BssHII, BstBI, BstUI, Cfr10I, ClaI, EagI, Eco52I, XmaIII, FauI, FseI, FspI, HaeII, HgaI, HhaI, HinP1I, HpaII, Hpy99I, HpyCH4IV, KasI, MluI, NaeI, NarI, NgoMIV, NotI, NruI, PaeR7I, PluTI, PmaCI, PmlI, PvuI, RsrII, SacII, SalI, SfoI, SgrAI, SmaI, SnaBI, TspMI and ZraI.

29. The method of claim 28, wherein the MSRE is selected from the group consisting of Aor13HI, BspMII, AccIII, Aor51HI, Eco47III, BspT104104, AsuII, NspV, Eco52I, XmaIII, PluTI, PmaCI, PmlI and RsrII.
